SEQUENCE LISTING (6 sequences)

SEQ ID NO: 1 - DNA, 18 bp, Artificial Sequence
  synthetic oligonucleotide primer
tggccctcctgcttcaga

SEQ ID NO: 2 - DNA, 18 bp, Artificial Sequence
  synthetic oligonucleotide primer
gtcctggcactggctgct

SEQ ID NO: 3 - DNA, 27 bp, Artificial Sequence
  synthetic oligonucleotide probe
  misc_feature 1: the 5′ residue is conjugated to a 6-FAM      fluorescent reporter dye
  misc_feature 27: the residue is conjugated to a TAMRA-Q      fluorescent dye quencher
catagatgtgtggagctggtgcctgga

SEQ ID NO: 4 - DNA, 20 bp, Artificial Sequence
  synthetic oligonucleotide primer
ggcaaattcaacggcacagt

SEQ ID NO: 5 - DNA, 19 bp, Artificial Sequence
  synthetic oligonucleotide primer
agatggtgatgggcttccc

SEQ ID NO: 6 - DNA, 26 bp, Artificial Sequence
  synthetic oligonucleotide probe
  misc_feature 1: the 5′ residue is conjugated to a 6-FAM fluorescent reporter dye
  misc_feature 26: the 3′ residue is conjugated to a TAMRA-Q fluorescent dye quencher
aggccgagaatgggaagcttgtcatc

(19) United States
(12) Patent Application Publication
(10) Pub. No.: US 2019/0083455 A1
(43) Pub. Date: Mar. 21, 2019

(54) METHODS AND COMPOSITIONS FOR THE MODULATION OF BETA-ENDORPHIN LEVELS

(71) Applicant: The General Hospital Corporation, Boston, MA (US)

(72) Inventors: David E. Fisher, Newton, MA (US); Kathleen Robinson, Boston, MA (US); Gillian Fell, Boston, MA (US); Rosa Veguilla, Cambridge, MA (US)

(21) Appl. No.: 16/202,076

(22) Filed: Nov. 27, 2018

Related U.S. Application Data
(62) Division of application No. 15/318,245, filed on Dec. 12, 2016.
(60) Provisional application No. 62/013,875, filed on Jun. 18, 2014.

(51) Int. Cl. A61K 31/352; A61K 31/573; A61K 38/22; A61K 31/522; A61K 31/4709; A61K 31/444; A61K 31/4015; A61K 31/222; A61K 31/167; A61K 31/137; A61N 5/06; A61K 9/00; A61K 38/33; A61K 38/29; A61K 38/16; A61K 38/04; A61K 31/5575

(52) U.S. Cl. CPC A61K 31/352; A61K 31/573; A61K 31/5575; A61K 31/522; A61K 31/4709; A61K 31/444; A61K 31/4015; A61K 31/222; A61K 31/167; A61K 31/137; Y02A 50/471; A61N 5/0616; A61N 2005/0661; A61K 9/0014; A61K 38/33; A61K 38/29; A61K 38/2271; A61K 38/164; A61K 38/046; A61K 38/043; A61K 38/2228

(57) ABSTRACT

Methods and compositions for treatment of pain, mood, or for the treatment of opiate withdrawal symptoms with the modulation of systemic beta-endorphin levels by the topical administration of cAMP elevating agents and/or dermal exposure to ultraviolet (UV) irradiation.

### CLAIM OF PRIORITY

This application is a divisional of U.S. patent application Ser. No. 15/318,245, filed Dec. 12, 2016, which is a § 371 National Stage Application of PCT/US2015/036399, filed Jun. 18, 2015, which claims the benefit of U.S. Provisional Patent Application Ser. No. 62/013,875, filed on Jun. 18, 2014. The entire contents of the foregoing are hereby incorporated by reference.

### FEDERALLY SPONSORED RESEARCH OR DEVELOPMENT

This invention was made with Government support under Grant Nos. R01-AR043369 and R01-CA150226-03 awarded by the National Institutes of Health. The Government has certain rights in the invention.

### TECHNICAL FIELD

This invention relates to the modulation of systemic beta-endorphin levels, and more particularly to methods and compositions for treatment of pain, mood, or the treatment of opiate withdrawal symptoms with the modulation of systemic beta-endorphin levels.

### BACKGROUND

Excessive UV exposure (e.g., indoor or outdoor tanning, two or more times per week) is associated with increased incidence of skin cancer. Systemic effects of cutaneous radiation exposure (e.g., sun-seeking and radiation-induced fatigue) are believed to include psychological or emotional reactions. Methods of reducing UV-seeking behavior have focused on educational measures to raise awareness of UV-associated skin cancer risk. Despite widespread awareness that UV exposure is a major risk factor for all common cutaneous malignancies, skin cancer incidence relentlessly increases by ˜3% per year (de Gruijl, 1999; Gandini et al., 2011; Robinson et al., 1997).

UV-seeking behavior is a recognized risk factor, but it is incompletely understood whether the popularity of sunbathing represents a biological addiction or an aesthetic preference for tanned skin. Studies have reported that many UV-seekers meet CAGE and DSM-IV criteria for a substance-related disorder with respect to UV (Harrington et al., 2011; Kourosh et al., 2010; Lazovich et al., 2010; Mosher and Danoff-Burg, 2010; Warthan et al., 2005). UV-seekers were capable of distinguishing between true UV and mock treatment in blind tanning bed experiments (Feldman et al., 2004), and two studies involving small cohorts of frequent tanners revealed that acute administration of the opioid antagonist naltrexone can induce withdrawal-like symptoms (Kaur et al., 2005; Kaur et al., 2006b). While a mechanism for such addiction has been lacking, these studies are consistent with the possibility of endogenous opioid-mediated addictive behavioral effects.

In the cutaneous response to UV exposure, epidermal keratinocytes respond to DNA damage via p53-mediated transcriptional induction of the proopiomelanocortin (POMC) gene (Cui et al., 2007). POMC is post-translationally cleaved into biologically active peptides, one of which is α-Melanocyte Stimulating Hormone (MSH) that mediates the tanning process by stimulating adjacent melanocytes to produce the brown/black pigment eumelanin (D'Orazio et al., 2006). The endogenous opioid β-endorphin is also post-translationally generated in skin by cleavage of the POMC pro-peptide in response to UV radiation (Cui et al., 2007; Skobowiat et al., 2011; Slominski and Wortsman, 2000). β-endorphin is the most abundant endogenous opioid, with basal plasma levels of 1 pM-12 pM (Bender et al., 2007; Fassoulaki et al., 2007; Leppaluoto et al., 2008), and intravenous administration of β-endorphin has been shown to cause analgesia (Tseng et al., 1976). β-endorphin binds with high affinity to the μ-opioid receptor (Schoffelmeer et al., 1991), although some evidence suggests that it may also act through other mechanisms that are, at present, incompletely characterized (Nguyen et al., 2012). Exogenous opioids with similar mechanisms are analgesic, and have reinforcing properties that make them addictive when administered systemically. Chronic opioid exposure results in tolerance (increasing dose requirement to achieve comparable efficacy) and physical dependence (opioid antagonism produces withdrawal). β-endorphin plays a role in analgesia (Ibrahim et al., 2005; Kastin et al., 1979) as well as in the reinforcement and reward that underlie addiction (Gianoulakis, 2009; Olive et al., 2001; Racz et al., 2008; Roth-Deri et al., 2003; Trigo et al., 2009).

### SUMMARY

This disclosure provides methods and compositions for mediating changes in endogenous beta-endorphin levels.

At least in part, the present invention is based on the discovery that repeated UV exposure produces an opioid receptor-mediated addiction due to elevations in circulating levels of β-endorphin, leading to increased nociceptive thresholds that are reversed by naloxone or ablated in β-endorphin null mice. At least in part, the present invention is also based on the discovery that the POMC-derived peptide, β-endorphin, is coordinately synthesized in skin, elevating plasma levels after low-dose UV.

In one aspect, the disclosure provides methods for treating, preventing or ameliorating opiate withdrawal in a subject (e.g., the treatment of symptoms associated with opioid-withdrawal), the method comprising topically administering to a subject in need of said treatment a composition comprising an effective amount of one or more cyclic-AMP (cAMP) elevating agents.

In another aspect, the disclosure provides methods for treating, preventing or ameliorating pain in a subject, the method comprising administering to a subject in need of such treatment a topical composition comprising a therapeutically effective amount of one or more cyclic-AMP (cAMP) elevating agents. The pain can be chronic or acute pain.

In yet another aspect, the disclosure provides methods for treating, preventing or ameliorating a mood disorder in a subject, the method comprising administering to a subject in need of such treatment a topical composition comprising a therapeutically effective amount of one or more cyclic-AMP (cAMP) elevating agents.

The disclosure also provides compositions (e.g., topical compositions) for use in the treatment of pain comprising one or more cyclic-AMP elevating agents.

In another aspect, the disclosure provides compositions (e.g., topical compositions) for use in the treatment of symptoms associated with opiate withdrawal comprising one or more cyclic-AMP elevating agents.

In another aspect, the disclosure provides compositions (e.g., topical compositions) for use in the treatment of a mood disorder comprising one or more cyclic-AMP elevating agents.

In some aspects of the invention, the subject has a Fitzpatrick Skin Type I, II or III.

The one or more cAMP elevating agents can be any agent capable of increasing the intracellular level of cAMP. In one embodiment, the cAMP elevating agent can be selected from the group consisting of forskolin, a forskolin derivative, amrinone, aminophylline hydrate, N6-2′-O-dibutyryl cAMP (Bu2cAMP), butein, caffeine, calmidazolium chloride, CART (61-102), cholera toxin, cicaprost, cilostamide, cilostazol, dbcAMP, (Des-Arg9,Leu8)-bradykinin, (Des-Arg9)-bradykinin, 2,6-dihydroxy-1,3-dimethylpurine, 1,3-dimethylxanthine, dobutamine, dopamine, dopexamine, DTLET, eledoisin, epinephrine, enoximone, etazolate hydrochloride, formoterol, glucocorticoid (dexamethasone), ibopamine, 4-(3-butoxy-4-methoxybenzyl)imidazolidin-2-one, imidazolium chloride, 1-[bis(4-chlorophenyl)methyl]-3-[2-(2,4-dichlorophenyl)-2-(2,4-dichlorobenzyloxy)ethyl]-1H-imidazolium chloride, 1-methyl-3-isobutylxanthine, isoproterenol, 3-isobutyl-1-methylxanthine, 8-methoxymethyl-3-isobutyl-1-methylxanthine, milrinone, α-neoendorphin, norepinephrine, neuropeptide Y fragment 22-36, papaverine hydrochloride, [Nle8,18, Tyr34]-parathyroid hormone (1-34) amide, pentoxyfilline, pertussis toxin (an AB5 protein), propentofylline, 3-methyl-1-(5-oxohexyl)-7-propylxanthine, prostaglandin E1 (PGE1), prostaglandin E2 (PGE2), prostaglandin E3 (PGE3), 3-isobutyl-1-methyl-2,6(1H,3H)-purinedione, quercetin dihydrate, rolipram, salbutamol, salmeterol, SKF 94836, [Cys3,6, Tyr8, Pro9]-substance P, theophylline, trifluoperazine dihydrochloride, TJBMX, and urotensin U. In some embodiments, the one or more cAMP elevating agents is a phosphodiesterase (PDE) 4 inhibitor. The PDE4 inhibitor can be selected form the group consisting of luteolin, cilomilast, mesembrine, rolipram, ibudilast, piclamilast, drotaverine, roflumisast, aminophylline, theophylline, 3-isobutyl-1-methylxanthine (IBMX) and caffeine.

In one embodiment, the one or more cAMP agents comprise forskolin and rolipram.

In some aspects, the methods disclosed herein further comprise irradiating the subject's skin with ultraviolet light, including, for example UVB light. In one embodiment, the ultraviolet light has a wavelength of between 280 and 320 nm, or between 300 and 315 nm.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-C (A) is a graph showing the effects of UV radiation on plasma beta-endorphin levels. A) Plasma β-endorphin in C57Bl6 mice receiving daily UV or Mock irradiation. Mice were treated twice a week with either Naloxone or Saline as indicated. Data are represented as the mean+/−SEM. 2way ANOVA analysis with Bonferroni's multiple comparisons test gives p<0.05 for both UV treated groups compared to both Mock treated groups (during UV treatment, days 14-42), and no significant effect of Naloxone treatment within either group. (B) and (C) are graphs showing the changes in pain thresholds over a 6-week regimen of chronic low-dose exposure to UV. B) von Frey thresholds and C) Hot Plate thresholds in chronically UV-irradiated and mock-irradiated C57Bl6 mice (mean+/−SEM). Half of each group was pre-treated with naloxone (10 mg/kg) 15 minutes prior to nociceptive testing, while the remainder received saline (n=10 per group). Analgesic thresholds were further monitored for 2 additional weeks after cessation of UV/Mock treatment. 2way ANOVA with Bonferroni's multiple comparisons test reveals p<0.0001 for the UV/Saline treated group compared to all other groups during UV treatment, days 9 to 39).

FIGS. 2A-C(A) is a graph showing Straub Tail scores over a 6-week regimen of chronic low-dose exposure to UV or mock exposure. A) Straub Tail in C57Bl6 mice over the course of 42 days of UV irradiation (n=13) or mock-irradiation (n=6). Data are represented as the mean+/−SEM, for days 10-37, p<0.0001 by 2way Anova analysis. (B) and (C) are graphs and photographic images showing the effects of naloxone on UV-induced Straub Tail. B) Straub Tail at day 17 before (Pre) and 15 minutes after (Post) injection of naloxone (n=7) or saline (n=6). Data are represented as the mean+/−SEM, p<0.001 by Student's t-test. C) Representative animals from each group in part (B). The beginning of black fur re-growth produces a patchy appearance.

FIGS. 3A-D. FIGS. 3A and 3B are graphs showing naloxone-induced somatic symptoms of opiate withdrawal in mice following UV exposure or mock exposure. A) Signs of opioid withdrawal in mice under experimental conditions described in FIG. 3. UV/saline (n=9), mock/saline (n=7), UV/naloxone (n=15), and mock/naloxone (n=7). Data are represented as the mean+/−SEM, *p<0.05 compared to UV/Saline group by 2way ANOVA with Bonferroni's multiple comparisons test. B) Conditioned place aversion testing in UV treated mice conditioned to the naloxone-paired box (black box) with an injection of naloxone or saline-paired box (white box) following 42 days of UV or mock treatment. Mice were permitted to freely move between naloxone-paired and saline-paired boxes prior to (pretest, n=8) and after 4 days of conditioning (test), and place preferences were assessed as change in time spent in the naloxone-paired box (postconditioning-preconditioning). Data are represented as the mean+/−SEM, and p values were generated by 2way ANOVA with Bonferroni's multiple comparisons test. FIG. 3C is a graph showing the effects of morphine on in mice following UV exposure or mock exposure. C) Morphine dose-response curves in mice following 42 days UV irradiation (n=31) or mock exposure (n=29). Data are represented as the mean+/−SEM, p<0.0001 by 2way ANOVA. FIG. 3D is a graph showing conditioned place preference testing in mice following UV exposure or mock exposure. D) Conditioned place preference testing in mice administered intravenous (i.v.) β-endorphin or saline through the tail vein. Mice were conditioned to β-endorphin (6) or saline (8) in the white box and saline in the black box. Place preferences were assessed as change in time spent in the white (β-endorphin-paired box) (postconditioning-preconditioning). Data are represented as the mean+/−SEM, p=0.0145 by Student's t-test.

FIGS. 4A-D. FIGS. 4A and 4B are graphs showing the changes in pain thresholds in wild type and β-endorphin−/− mice following UV exposure. A) von Frey test and B) thermal analgesic thresholds in wild type (n=11) and β-endorphin−/− (n=13) mice over 35 day UV exposure. Data are represented as the mean+/−SEM, *p<0.05 by 2way ANOVA with Bonferroni's multiple comparisons test. FIGS. 4C and 4D are graphs showing naloxone-induced somatic symptoms of opiate withdrawal in wild type and beta-endorphin−/− mice following UV exposure or mock exposure. C) Signs of naloxone precipitated opioid withdrawal in control and β-endorphin null mice after 6 weeks of UV exposure. Data are represented as the mean+/−SEM, *p<0.0001 compared to β-endorphin−/− naloxone group by 2way ANOVA with Bonferroni's multiple comparisons test. D) Conditioned place aversion testing in UV treated control and β-endorphin null mice conditioned to the naloxone-paired box (black box) with an injection of naloxone or saline. All mice were conditioned to saline in the white box; n>10 for all groups. Mice were permitted to freely move between naloxone-paired and saline-paired boxes prior to and after 4 days of conditioning, and place preferences were assessed as change in time spent in the naloxone-paired box (postconditioning-preconditioning). Data are represented as the mean+/−SEM, p values were generated by 2way ANOVA with Bonferroni's multiple comparisons test.

FIGS. 5A-D. FIG. 5A is a photographic image showing representative K14cre and p53fl/fl K14cre mice following exposure to UV. A) Representative K14cre and p53fl/fl K14cre mice after 4 weeks of daily UV treatment. FIG. 5B is a graph showing the effects of UV radiation on plasma β-endorphin levels in K14cre and p53fl/fl K14cre mice receiving chronic low-dose UV radiation. B) Plasma β-endorphin in mice in K14cre and p53fl/fl K14cre mice receiving daily UV irradiation. Data are represented as the mean+/−SEM, *p<0.05 by 2way ANOVA analysis with Bonferroni's multiple comparisons test. FIG. 5C is a graph showing the changes in pain thresholds in K14cre and p53fl/fl K14cre mice. C) Mechanical analgesic thresholds in K14cre (n=10) and p53fl/fl K14cre (n=9) mice over 13 days UV exposure. Data are represented as the mean+/−SEM, *p<0.05 by 2way ANOVA analysis with Bonferroni's multiple comparisons test. FIG. 5D is a graph showing naloxone-induced conditioned place aversion in K14cre and p53fl/fl K14cre mice. D) K14cre and p53fl/fl K14cre mice were conditioned to naloxone in the black box after 3 weeks of daily UV exposure. Place preferences were assessed as change in time spent in the naloxone-paired box (postconditioning-preconditioning). Data are represented as the mean+/−SEM, p=0.0317 by Student's t-test. The change in time spent in the black box was not significant when the postconditioning and preconditioning times were compared by Student's t-test (p=0.26).

FIGS. 6A-D are graphs showing effects of forskolin on MOMC and MitF expression. B16, B16-F0, Melan-a and PAM212 mouse melanoma cell lines (upper panel) or Malme-3M, UAC257 and UACC62 human melanoma cell lines (below panel) were treated with 20 uM final concentration of forskolin as shown. Fold expression change of POMC (right) and Mitf (left) after forskolin treatment are shown. Graphs represent biological triplicates.

FIG. 7 is a graph demonstrating the effect of MITF expression on POMC expression levels in human melanoma. Human melanoma Malme-3M cell line was transfected with Si-Mitf or Si-Control for 48 hrs. Cells were harvested and mRNA extraction was followed by POMC qPCR analysis. Graphs represent biological triplicates.

FIGS. 8A and 8B are graphs showing the effect of topical forskolin treatment on β-endorphin levels in both K14 e/e as well as e/e female mice. A) Basal β-endorphin levels are shown at time 0. Mice were treated with forskolin (80 μL, 20%-Forskolin extract) daily. After 5 weeks, forskolin treatment was stopped and recollection of β-endorphin plasma levels was continued for 1 more week. Black circle shows start point of forskolin treatment and black arrow shows the end of treatment. β-endorphin levels were measured by a competitive radioactive assay. Data represents 10 mice per condition.

FIG. 9 is a photograph demonstrating the effect of topical forskolin on pigmentation in K14-e/e mice. Mice were treated with 80 μL, 20%-Forskolin extract daily for four weeks, after which the picture was taken. From left to right: e/e-Forskolin, e/e vehicle control, K14-e/e-Forskolin and K14-e/e-vehicle control. The color observed in the e/e-Forskolin treated mice is not pigmentation but the color of the Forskolin extract.

FIGS. 10A and 10B are graphs showing upregulation of β-endorphin following forskolin (Fsk) topical treatment in K14-e/e male mice. A) Basal β-endorphin levels are shown at time 0. Mice were pre-treated with vehicle for two weeks and then treated with forskolin (80 μL, 20%-forskolin extract) daily. After 5 weeks, forskolin treatment was stopped and recollection of β-endorphin plasma levels was continued for 1 more week. Black circle shows start point of forskolin treatment and black arrow shows the end of treatment. β-endorphin levels were measured by a competitive radioactive assay. Data represents 10 mice per condition.

FIGS. 11A and 11B are graphs showing the effect on β-endorphin levels following forskolin (Fsk) and rolipram (Rp) treatment in K14-e/e female mice. A) Basal β-endorphin levels are shown at time 0. Mice were treated with forskolin (40 μL, 20%-Forskolin extract)+rolipram (40 μL, 10 μM) daily starting 2 days after the basal point was taken. Mice were treated for 4 weeks and β-endorphin levels were monitored weekly. Data represent the result of 5 mice per condition. B) Opioid dependency of these mice was verified by naloxone treatment.

### DETAILED DESCRIPTION

This disclosure is based, in part, on the discovery that repeated UV exposure produces an opioid receptor-mediated addiction due to elevations in circulating β-endorphin levels, leading to increased nociceptive thresholds that are reversed by naloxone or ablated in β-endorphin null mice.

### Definitions

The term “ionizing radiation,” as used herein, refer to energy sources that induce DNA damage, such as gamma-rays, X-rays, UV-irradiation, microwaves, electronic emissions, particulate radiation (e.g., electrons; protons, neutrons, alpha particles, and beta particles), and the like. An irradiating energy source may be carried in waves or a stream of particles or photons. Further, an irradiating energy source has sufficient energy or can produce sufficient energy via nuclear interactions to produce ionization (gain or loss of electrons). Ionizing radiation can be directed at target tissues (e.g., a cancer cell population) for purposes of reducing the viability of such tissues. Ionizing radiation can be delivered from an external source or from an internal implant at the site of the target tissue. When using X-ray, clinically relevant doses are preferred, and these may be applied in single doses or fractionated, as is known in the art.

The terms “gray” or “Gy” refer to a unit of measurement for the amount of ionizing radiation energy absorbed by body tissues. A gray is equal to 100 rad and is now the unit of dose. A “centigray” or “cGy” is equal to 1 rad.

The ultraviolet region (UV region) is a region of the electromagnetic spectrum adjacent to the low end of the visible spectrum. The UV region extends between 100-400 nm, and is divided into 3 sub regions: the UVA region (320-400 nm), the UVB region (280-320 nm), and the UVC region (100-280 nm). In the literature, the boundaries of these regions are sometimes slightly varied from these numbers.

The term “minimal erythemal dose” (MED) refers to a quantity of radiation associated with the erythemal potential due to exposure to UV radiation. An MED is defined as the radiant exposure of the UV radiation that produces a just noticeable erythema on previously unexposed skin. The radiant exposure to monochromatic radiation at around 300 nm with the maximum spectral efficacy, which is required for erythema, corresponds to an approximate dose of 200 to 2000 J/m2 depending on the skin type (i.e., fair vs. dark skin).

The terms “patient” or “subject” are used throughout the specification to describe an animal, human or non-human, rodent or non-rodent, to whom treatment according to the methods of the present invention is provided. Veterinary and non-veterinary applications are contemplated. The term includes, but is not limited to, birds, reptiles, amphibians, and mammals, e.g., humans, other primates, pigs, rodents such as mice and rats, rabbits, guinea pigs, hamsters, cows, horses, cats, dogs, sheep and goats. Typical patients include humans, farm animals, and domestic pets such as cats and dogs.

Term “addictive” refers to a substance, including but not limited to an opioid, that has the potential to cause physical dependence and/or psychological dependence in a subject to whom it is administered. A “psychological dependence” is a psychological condition that manifests as an overpowering compulsion to continue taking an addictive substance; “physical dependence” is a state of physiologic adaptation to an addictive substance, which may increase in intensity when tolerance develops and requires increased dosage and duration of use of the addictive substance.

Other definitions appear in context throughout this disclosure.

### Methods of Treatment

In some aspects, this disclosure provides methods and compositions for treating, preventing or ameliorating pain. The pain treated can be acute pain or chronic pain. The terms “chronic pain” and “acute pain” incorporate their common usages; subjective (e.g., clinical diagnosis) and objective means (e.g., laboratory tests, PET) to determine the presence of chronic pain and/or acute pain, and to distinguish between these two distinct categories of pain, are described in detail, below. Distinguishing chronic from acute pain is always subjective and can have physiologic, pathophysiologic, psychologic, emotional, and affective dimensions. Acute pain, such as occurs after surgery or trauma, comes on suddenly and lasts for a limited time. Acute pain is a direct response to disease or injury to tissue, and typically subsides when the disease or injury is adequately treated. Chronic pain, on the other hand, is pain that persists for an extended period of time (e.g., at least a week, at least a month, at least a year, or longer), sometimes even after a known precipitating cause no longer exists. Chronic pain may result from various abnormal or compromised states (e.g., diseased), including but not limited to osteoarthritis, rheumatoid arthritis, psoriatic arthritis, back pain, cancer, injury or trauma. Common types of chronic pain include back pain, headaches, arthritis, cancer pain, and neuropathic pain resulting from injury to nerves.

As used herein, the phrase “effective to treat pain” means effective to ameliorate or minimize the clinical impairment or symptoms resulting from the pain (e.g., by diminishing any uncomfortable, unpleasant, or debilitating sensations experienced by the subject). The amounts of UV exposure and/or the cAMP elevating agent will vary depending on the particular factors in each case, including the type of pain, the location of the pain, the subject's weight, the severity of the subject's condition, the agent used, and the route of administration.

In some aspects, this disclosure provides methods and compositions for treating, preventing or ameliorating mood disorders (e.g., personalities) in a patient in need thereof. The term “mood disorder” refers to any psychological disorder characterized by the elevation or lowering of a person's mood. In a subject who experiences a mood disorder, the subject's emotional state or mood is distorted or inconsistent with their circumstances. Mood disorders are classified (see, e.g., the Diagnostic and Statistical Manual of Mental Disorders (DSM) IV or V (American Psychiatric Association)) as Depressive Disorders and Bipolar Disorders. Depressive Disorders include Major Depressive Disorder (single or recurrent) and Dysthymic Disorder. Bipolar Disorders comprise: BD I (which presents with an alternation of episodes of major depression and recurrent episodes of mania); BD II (which is made up of episodes of major depression and recurrent hypomanias); and Cyclothymic Disorder (for at least two years several hypomanic and depressive episodes which must not be major). Further, a Mixed Episode is when symptoms of major depression and mania are present in the same episode. These disorders may sometimes have a rapid-cycling course, marked by the presence of at least four cycles per year (a cycle equals one episode of depression followed by mania, or vice versa), which is very often resistant to current treatments. In some embodiments, the methods and compositions disclosed herein treat mood disorders as the disregulation of any affect, including sadness, anger, joy, anxiety, fear, guilt, and shame. In addition, this disclosure provides methods and compositions for treating, preventing or ameliorating mood spectrum disorder.

In some aspects, this disclosure provides methods and compositions for treating, preventing or ameliorating symptoms and consequences of premenstrual syndrome, including but not limited to cramping, breast tenderness, headaches, backaches, bloating, irritability, depression and skin problems. In more severe cases, patients present with Premenstrual Dysphoric Disorder (PMDD), a condition in which severe depression, irritability, and tension manifest before menstruation.

In some aspects, this disclosure provides methods and compositions for treating, preventing or ameliorating opioid-withdrawal (e.g., the treatment of symptoms associated with opioid-withdrawal) in a subject in need thereof. As used herein, the term “opioid” refers to a natural or synthetic compound that binds to specific opioid receptors in the central nervous system (CNS) and peripheral nervous system (PNS) of a subject, and has agonist (activation) or antagonist (inactivation) effects at these receptors. Opioids may be endogenous (originating within the subject) or exogenous (originating outside of the subject). Opioids that have agonist (activating) effects at inhibitory opioid receptors produce analgesia. In addition, at high doses they may elicit narcosis (a non-specific and reversible depression of function of the CNS or PNS, marked by insensibility or stupor). Thus, such opioid agonists are often referred to as “narcotics,” whereas opioid antagonists (e.g., naloxone, naltrexone) are non-narcotic. Examples of opioid compounds include, without limitation, opioid alkaloids (e.g., the agonists, morphine and oxycodone, and the antagonists, naloxone and naltrexone) and opioid peptides (e.g., dynorphins, endorphins, and enkephalins).

Opiates are a class of drugs that are commonly prescribed to treat pain. Prescription opiates include Oxycontin (oxycodone), Vicodin (hydrocodone and acetaminophen), Dilaudid (hydromorphone), and morphine. Certain illegal drugs, such as heroin, are also opiates. Methadone is an opiate that is often prescribed to treat pain, but may also be used to treat withdrawal symptoms in people who have become addicted to opiates.

Opiate withdrawal refers to the wide range of symptoms that occur after stopping or dramatically reducing opiate drugs after heavy and prolonged use (several weeks or more). Opioid withdrawal reactions are very uncomfortable but are not life threatening. Symptoms of opioid withdrawal are well known and include pronounced intensity of (i) psychic feelings such as anxiety or fear, and cravings for opiate, (ii) general autonomic signs such as yawning, perspiration, lacrimation (eyes tearing up), rhinorrhea, mydriasis, palpitation, hot and cold “flashes” and gooseflesh, (iii) neuromuscular signs such as restlessness, aching bones and muscles, tremors and weakness, (iv) gastrointestinal signs such as abdominal cramps, diarrhea, nausea vomiting, and loss of appetite and (v) sleep disturbances such as difficulty in falling asleep and interrupted sleep.

As used in this context, to “treat” means to ameliorate at least one symptom or complication associated with pain, opioid withdrawal or a mood disorder as described herein.

An “effective amount” is an amount sufficient to effect beneficial or desired results. For example, a therapeutic amount is one that treats the disorder or achieves a desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including, but not limited to, the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity, and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are typically preferred. While compounds that exhibit toxic side effects may be used, care should be taken to design a delivery system that targets such compounds to the site of affected tissue to minimize potential damage to uninfected cells and, thereby, reduce side effects.

The data obtained from cell culture assays and animal studies can be used in formulating a range of dosages for use in humans. The dosage of such compounds lies preferably within a range of circulating concentrations that include the ED50 with little or no toxicity. The dosage may vary within this range depending upon the dosage form employed and the route of administration utilized. For any compound used in the methods of the inventions described herein, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. β-endorphin levels in plasma may be measured, for example, by high performance liquid chromatography.

In some aspects, the methods and compositions disclosed herein include administering a therapeutically effective amount of a cAMP elevating agent to a subject who is in need of, or who has been determined to be in need of, such treatment.

The terms “cAMP elevating agent” and “cAMP enhancing agent” are used interchangeably and refer to agents (e.g., inorganic and organic compounds, proteins and peptides, polysaccharides and other sugars, lipids, and nucleic acid sequences having therapeutic activities) capable of increasing the intracellular level of cAMP. The level of intracellular cAMP is increased when a higher amount, such as for example at least 2% more, at least 5% more, at least 10% more, at least 20% more, at least 30% more, at least 50% more, at least 75% more, at least 100% more, or at least 1000% more cAMP is present in a cell that has been contacted with the agent as compared to a cell not contacted with the agent. An increase in intracellular cAMP levels can be achieved for example by inhibition of the activity of phosphodiesterase and/or by activating adenylate cyclase. Preferred amounts of cAMP elevating agent to be employed are between about 0.0001 and about 100 mM, between about 0.001 and about 90 mM, between about 0.01 and about 80 mM, between about 0.01 and about 50 mM, between about 0.1 and about 40 mM, between about 1 and about 20 mM, between about 0.001 and about 10 mM, between about 0.01 and about 1 mM, or between about 0.05 and about 0.5 mM.

cAMP elevating agents are well known in the art and some are disclosed herein. Non-limiting examples include agents that directly enhance cAMP (e.g., forskolin and derivatives thereof including, for example, forskolin derivatives disclosed in U.S. Pat. Nos. 4,954,642; 4,871,764; 5,550,864; 5,789.439;), cAMP selective (i.e., specific) phosphodiesterase (PDE) 4 inhibitors (e.g., apremilast, rolipram, mesembrine, mesembenone, ibudilast, piclamilast, luteolin, drotaverine, diazepam, cilomilast, Arofylline, Atizoram, Denbutylline, Etazolate, Etazolate, Filaminast, Glaucine, HT-0712, ICI-63197, Irsogladine, Piclamilast, Ro20-1724, RPL-554, YM-976 and roflumilast, WO 2013021021) and non-specific cAMP PDE inhibitors (e.g., methylxanthines as aminophylline, theophylline, isobutylmethylxanthine, 3-isobutyl-1-methylxanthine (IBMX), caffeine, and similarly-acting agents). Additional cAMP elevating agents include, for example, selected from the group consisting of forskolin, a forskolin derivative, amrinone, aminophylline hydrate, N6-2′-O-dibutyryl cAMP (Bu2cAMP), butein, caffeine, calmidazolium chloride, CART (61-102), cholera toxin, cicaprost, cilostamide, cilostazol, dbcAMP, (Des-Arg9,Leu8)-bradykinin, (Des-Arg9)-bradykinin, 2,6-dihydroxy-1,3-dimethylpurine, 1,3-dimethylxanthine, dobutamine, dopamine, dopexamine, DTLET, eledoisin, epinephrine, enoximone, formoterol, glucocorticoid (dexamethasone), ibopamine, 4-(3-butoxy-4-methoxybenzyl)imidazolidin-2-one, imidazolium chloride, 1-[bis(4-chlorophenyl)methyl]-3-[2-(2,4-dichlorophenyl)-2-(2,4-dichlorobenzyloxy)ethyl]-1H-imidazolium chloride, 1-methyl-3-isobutylxanthine, isoproterenol, 3-isobutyl-1-methylxanthine, 8-methoxymethyl-3-isobutyl-1-methylxanthine, milrinone, α-neoendorphin, norepinephrine, neuropeptide Y fragment 22-36, papaverine hydrochloride, [Nle8,18, Tyr34]-parathyroid hormone (1-34) amide, pentoxyfilline, pertussis toxin (an AB5 protein), propentofylline, 3-methyl-1-(5-oxohexyl)-7-propylxanthine, prostaglandin E1 (PGE1), prostaglandin E2 (PGE2), prostaglandin E3 (PGE3), 3-isobutyl-1-methyl-2,6(1H,3H)-purinedione, quercetin dihydrate, salbutamol, salmeterol, SKF 94836, [Cys3,6, Tyr8, Pro9]-substance P, theophylline, trifluoperazine dihydrochloride, TJBMX, and urotensin U. In some embodiments, the cAMP elevating agent is forskolin. In some embodiments, the cAMP elevating agent is not a cGMP inhibitor of PDE5. In some embodiments, the cAMP elevating agent is forskolin in combination with at least one additional cAMP elevating agent.

In some aspects, the methods disclosed herein include administering an effective amount of UV irradiation (e.g., UVB light) to a patient's skin. Various UV radiation sources can be used in accordance with the present invention to deliver a therapeutically effective amount of UV light to a patient's skin. Skin has been classified into different skin types, which present with different responses to environmental abuses. Fitzpatrick skin types may be determined as set forth in Fitzpatrick, Thomas B.: Soleil et Peau. J Med Esthet 1975; 2:33034. The scale ranges from type I (ivory white skin) to type VI (dark brown skin) and identifies skin type based on its reaction to UV light. Skin of color can be classified as skin types IV-VI.

Methods to administer UV radiation are well known in the art. Either pulsed or continuous wave (“CW”) lasers can be used. The therapeutic UV radiation useful in the present invention will typically range from about 280 nanometers to about 320 nanometers, or from about 300 nanometers to about 315 nanometers. The energy of the UV radiation can be about 5 J/cm² per pulse or less for pulsed lasers, or a total dose of between about 10 J/cm² to about 1000 J/cm², between about 20 J/cm² to about 900 J/cm², between about 30 J/cm² to about 800 J/cm², between about 40 J/cm² to about 700 J/cm², between about 50 J/cm² to about 600 J/cm², between about 60 J/cm² to about 1000 J/cm², between about 70 J/cm² to about 900 J/cm², between about 80 J/cm² to about 800 J/cm², between about 90 J/cm² to about 700 J/cm², between about 100 J/cm² to about 600 J/cm², between about 200 J/cm² to about 500 J/cm², between about 300 J/cm² to about 400 J/cm², about 20 J/cm², about 30 J/cm², about 40 J/cm², about 50 J/cm², about 60 J/cm², about 70 J/cm², about 80 J/cm², about 90 J/cm², about 100 J/cm², about 200 J/cm², about 300 J/cm², about 400 J/cm², 500 J/cm², about 600 J/cm², about 700 J/cm² about 800 J/cm², about 900 J/cm², or about 1000 J/cm².

An effective amount is a dosage of the therapeutic agent sufficient to provide a medically desirable result. The effective amount will vary with the particular condition being treated, the age and physical condition of the subject being treated, the severity of the condition, the duration of the treatment, the nature of the concurrent therapy (if any), the specific route of administration and the like factors within the knowledge and expertise of the health care practitioner. It should be understood that the therapeutic agents of the invention are used to treat and/or prevent pain, opioid withdrawal or a mood disorder as described herein. Thus, in some cases, they may be used prophylactically in human subjects at risk of developing pain, opioid withdrawal or a mood disorder as described herein. Thus, in some cases, an effective amount is that amount which can lower the risk of, slow or perhaps prevent altogether the development of the pain, opioid withdrawal or mood disorder as described herein. It will be recognized that when the therapeutic agent is used in acute circumstances, it is used to prevent one or more medically undesirable results that typically flow from such adverse events.

Methods for selecting a suitable treatment and an appropriate dose thereof will be apparent to one of ordinary skill in the art.

### Pharmaceutical Compositions and Methods of Administration

The methods described herein include the manufacture and use of pharmaceutical compositions. Also included are the pharmaceutical compositions themselves.

In accordance with the method of the present invention, the cAMP elevating agent may be administered to a human or animal subject by known procedures, including, without limitation, transmucosal, transdermal, intracutaneous, intradermal, intramuscular, and intraperitoneal (particularly in the case of localized regional therapies) administration. Preferably, the cAMP elevating agents of the present invention are administered topically.

Pharmaceutical compositions are typically formulated to be compatible with their intended route of administration. Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Administration of a therapeutic compound as described herein can be by topical, transmucosal, or transdermal means. Transdermal (or other systemic) delivery, such as oral or intravenous, would be utilized in order to effect systemic upregulation of endorphin. For transdermal administration, formulations of the cAMP elevating agent may be combined with skin penetration enhancers, which increase the permeability of the skin to the agent and the inactivator, and permit the agent and the inactivator to penetrate through the skin and into the bloodstream. Such penetrants are generally known in the art, and include, for example, detergents, bile salts, fusidic acid derivatives, propylene glycol, polyethylene glycol, isopropanol, ethanol, oleic acid, N-methylpyrrolidone, and the like, for transmucosal administration. Transmucosal administration can be accomplished through the use of nasal sprays or suppositories. For transdermal administration, the active compounds are formulated into ointments, salves, gels, or creams as generally known in the art.

In some embodiments, compositions comprising a cAMP elevating agent for topical application can further comprise pharmaceutically acceptable carriers or vehicles and any optional components. A number of such cosmetically acceptable carriers, vehicles and optional components are known in the art and include carriers and vehicles suitable for application to skin (e.g., sunscreens, creams, milks, lotions, masks, serums, etc.), see, e.g., U.S. Pat. Nos. 6,645,512 and 6,641,824. In particular, optional components that may be desirable include, but are not limited to absorbents, anti-caking agents, anti-foaming agents, anti-oxidants, binders, buffering agents, bulking agents, chelating agents, colorants, dyes, essential oils, film formers, fragrances, humectants, hydrocolloids, light diffusers, opacifying agents, particulates, pH adjusters, sequestering agents, skin conditioners/moisturizers, skin feel modifiers, skin protectants, skin sensates, skin treating agents, kin soothing and/or healing agents, sunscreen actives, topical anesthetics, vitamin compounds, and combinations thereof.

Pharmaceutical compositions suitable for injectable use can include sterile aqueous solutions (when the composition is water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion or by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to select cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

The invention is further described in the following examples, which do not limit the scope of the invention described in the claims.

### Experimental Procedures for Examples 1-5

The following materials and methods were used in Examples 1-5.

### Mice.

All mice used were on a C57Bl/6 background. For select experiments, mice with homozygous deletion of the C-terminus of the POMC gene, resulting in lack of β-endorphin (β-endorphin−/−) (Rubinstein et al., 1996), and mice with a foxed allele of p53 (Marino et al., 2000) and a Keratin 14 promoter driven Cre recombinase strain (Dassule et al., 2000) were used.

### UV Irradiation and Blood Draws.

Mice were dorsally shaved two days prior to the start of radiation exposure, then exposed to 50 mJ/cm²/day of UVB, an empirically determined sub-erythematic dose, 5 days per week (Monday-Friday) for 6 weeks. If there were patches of fur re-growth, mice were re-shaved once every two weeks.

For blood draws, mice were placed in a standard restrainer and tail vein blood was collected in EDTA microvette tubes containing 0.6TIU aprotinin. Mice underwent blood draws prior to the start of radiation exposure, once per week during the radiation exposure regimen, and once per week for two weeks following cessation of the UV regimen. Blood was drawn in the mornings prior to radiation exposure on Fridays.

Tubes of collected blood were maintained on ice until centrifugation at 3500 RPM for 20 minutes at 4° C. Plasma was isolated and samples were stored at −80° C. until β-endorphin measurement. β-endorphin was quantified by radioimmunoassay (Phoenix Pharmaceuticals, Burlingame, Calif.).

### Straub Tail Measurement.

Straub Tail measurement was performed as described (Bilbey et al., 1960). Scoring was on a scale of 0-2 according to the angle of elevation of the tail from the horizontal plane (0=tail relaxed and no elevation; 1=tail is rigid and elevated 1-10° from horizontal; 1.5=11-45° elevation and rigidity at the base of the tail; 2=46-90° elevation and rigidity at the base of the tail). At each time point, each mouse was scored every 10 seconds for 1 minute and the final score was the average of these six values. Mice undergoing the six-week UV exposure regimen or mock treatment were scored prior to the start of the regimen, once per week during the regimen, and once per week for 2 weeks following cessation of UV/mock treatment. On day 23 of the regimen, after weekly Straub Tail scoring, mice were injected (intraperitoneal (ip)) with either 10 mg/kg naloxone hydrochloride (Sigma, St. Louis, Mo.) or saline. Mice underwent Straub Tail scoring again 15 minutes following injection.

### Analgesic Threshold Testing.

Mice underwent mechanical and thermal analgesic testing during UV/mock treatment regimens using the von Frey test (Kwan et al., 2006) and the hot plate test respectively (Mogil et al., 1999). In the von Frey test, mice were placed in individual enclosures on an elevated wire mesh rack and the plantar surface of the left hind paw was serially poked with fibers of increasing tensile strength (10 times per fiber at a rate of 1/second) until a paw withdrawal response was elicited on 2/10 pokes. In the hot plate test, mice were placed on a 52° C. hot plate and time to response (paw flutter, paw licking, jumping) was measured.

Mice were habituated to the wire mesh rack for 30 minutes per day and to the hot plate at room temperature for 2 minutes per day for 3 days, prior to measuring baseline nociceptive thresholds. Mice underwent nociceptive testing twice per week on non-consecutive days during and for two weeks following cessation of UV/mock treatment. Mice received an injection (ip) of 10 mg/kg naloxone or saline 15 minutes prior to nociceptive testing.

### Somatic Symptoms of Opiate Withdrawal.

Mice that had undergone 6 weeks of daily UV exposure or mock exposure were injected (ip) with either 2 mg/kg naloxone or saline, and signs of opioid withdrawal were tabulated as described (Olson et al., 2006). Mice were observed in an open-topped Plexiglas® 30 cm×15 cm×15 cm rectangular container for 25 minutes following each injection, and signs of opioid withdrawal were tabulated. Wet dog shake, teeth chatter, and bouts of grooming were measured as occurrence in each 15-second interval. Individual rearing events were counted. Number of fecal pellets at the end of the 25-minute interval was used to quantify diarrhea.

### Conditioned Place Aversion Testing.

The apparatus used consisted of a box with black interior and dim lighting and a box with white interior and bright lighting connected by a smaller gray “neutral” box, and procedures were followed as described (Skoubis et al., 2001; Weitemier and Murphy, 2009). Mice that had undergone 6 weeks of daily UV exposure or mock exposure were tested for baseline place preferences prior to conditioning (10-minute testing time per mouse). Over the following 4 days, conditioning took place in which mice were either conditioned with naloxone (10 mg/kg ip injection) or saline (ip injection) in the black box, and all animals were conditioned with saline (ip injection) in the white box. Conditioning time in each box was 30 minutes following injection. For each animal there were four hours between conditioning in one box and conditioning in the other box. On the day following the final day of conditioning, place preferences were again tested (post-conditioning, 10-minute testing time per mouse).

### Morphine Cross-Tolerance Testing.

Morphine dose-response curves in the hot plate test were measured as described (Mao et al., 2000) in mice that had undergone 6 weeks of UV exposure or mock treatment. Morphine was injected at a starting dose of 0.02 mg/kg ip, and was increased logarithmically in cumulative dose increments of 0.3 log units. Thermal analgesic thresholds were tested 15 minutes after each morphine injection until there was failure to respond in the hot plate test (cutoff time was 20 seconds) or until there was no change in response time from one dose to the next. There were 30 minutes between injections, and 30 minutes between hot plate testings for each mouse. Percent of maximal effect was calculated based on the equation: (test latency-baseline latency)/(maximal latency-baseline latency)×100% (Mao et al., 2000).

### Example 1: Systemic β-Endorphin Elevations Following Chronic UV Exposure

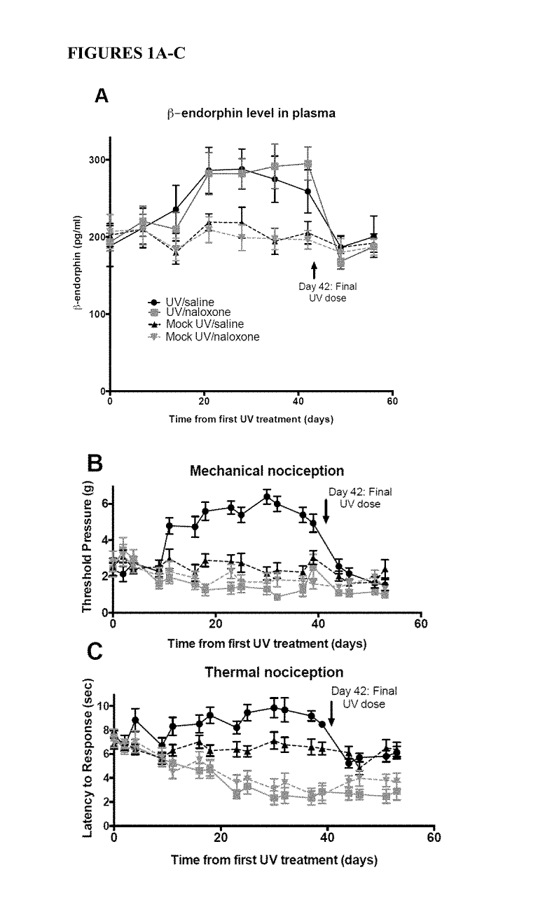

We developed a UV-exposure mouse model in which dorsally-shaved mice received a dose of 50 mJ/cm² of UVB, 5 days per week for 6 weeks, an empirically-derived sub-erythemic dose which is approximately equal to 20-30 minutes of ambient midday sun exposure in Florida during the summer for a fair-skinned person of average tanning ability (Fitzpatrick skin phototypes 2-3) (D'Orazio et al., 2006; Technology-Planning-and-Management-Corporation, 2000; US-EPA, 1994). After one week, significant elevations in circulating plasma β-endorphin were observed (FIG. 1A). Circulating β-endorphin levels remained elevated for the duration of the 6-week exposure regimen and returned within 7 days to near baseline levels after cessation of UV exposure. No significant changes in plasma β-endorphin were observed in mock UV-treated mice (FIG. 1A). Analgesic thresholds can be increased by peripheral administration of exogenous opioids or β-endorphin (Kastin et al., 1979). We quantified mechanical and thermal nociceptive thresholds over six weeks of daily UV exposure. Mechanical nociception was measured by the von Frey test (Kwan et al., 2006), which exposes fibers of increasing tensile strength to the plantar paw surface to elicit a paw withdrawal response. Thermal nociception was tested using the hot plate (52° C.) test (Mogil et al., 1999) in which time to response (paw licking, paw flutter, or jumping) was measured. UV-irradiated mice exhibited significant increases both in mechanical (FIG. 1B) and thermal (FIG. 1C) nociceptive thresholds. These elevated analgesic thresholds paralleled the UV-induced elevations in plasma β-endorphin (FIG. 1A). Mock-treated control mice displayed no significant elevations in pain thresholds (FIG. 1B and FIG. 1C). Treatment with naloxone, an opioid antagonist, 15 min prior to analgesic testing suppressed the UV-induced increases in mechanical and thermal nociceptive thresholds (FIG. 1B and FIG. 1C) despite maintained elevations in plasma β-endorphin (FIG. 1A). These data demonstrate opioid receptor mediated analgesia as a consequence of UV, that parallels the elevation of circulating blood β-endorphin levels.

### Example 2: Quantifiable Opioid-Mediated Behaviors Occur with Chronic UV Exposure

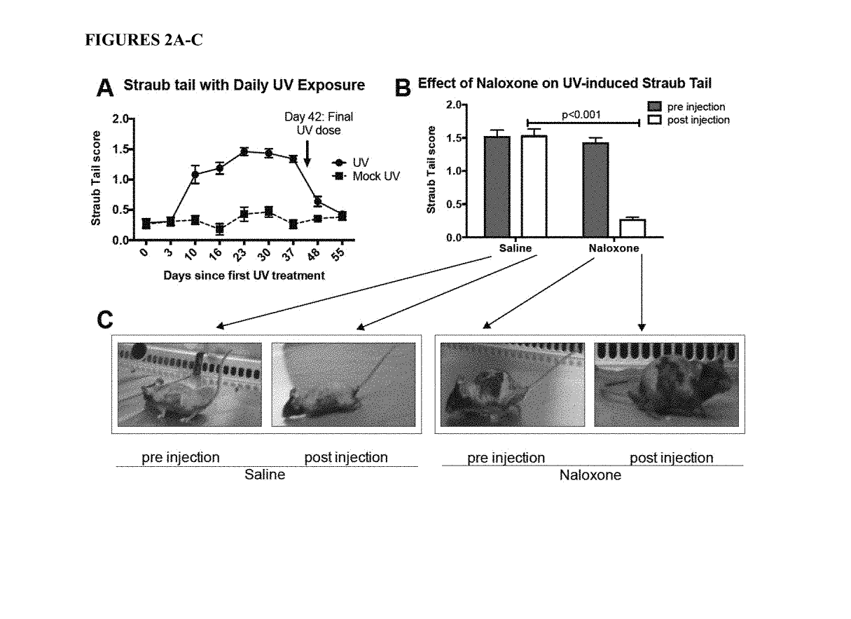

Exogenous opioids produce a dose-dependent, μ-opioid receptor-mediated contraction of the sacrococcygeus dorsalis muscle at the tail base in rodents, resulting in rigidity and elevation of the tail, a phenomenon called “Straub Tail” (Bilbey et al., 1960). Straub Tail was evident in UV-irradiated mice by the second week of daily UV exposure, persisted for the six-week exposure regimen, and diminished over two weeks after cessation of UV exposure (FIG. 2A). Treatment with the opioid antagonist naloxone (day 23 of the UV exposure regimen) reversed the Straub Tail phenotype (FIG. 2B, FIG. 2C).

### Example 3: Opioid Tolerance and Physical Dependence after Chronic UV Exposure

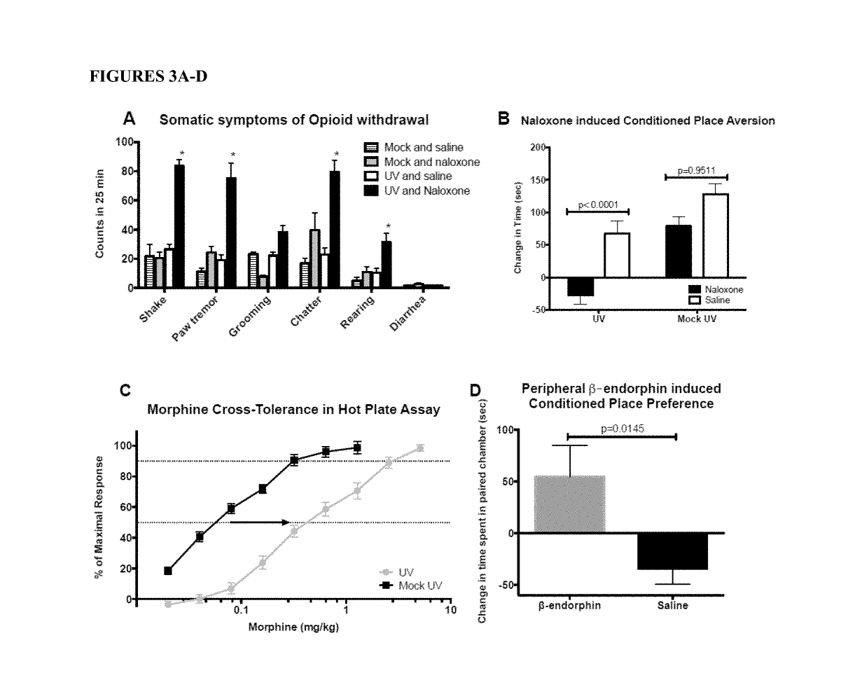

We next asked whether chronic UV exposure may be accompanied by detectable opioid dependence, in which opioid cessation or antagonism produces withdrawal symptoms, and tolerance in which increasing doses are required to achieve comparable analgesia (Drdla et al., 2009). Following chronic daily UV exposure, administration of naloxone elicited many of the classic murine signs of opioid withdrawal (wet dog shake, paw tremor, teeth chatter, rearing) (Olson et al., 2006) (FIG. 3A).

Because the magnitude of the measured withdrawal symptoms, while significant, was smaller than that commonly observed with exogenously administered opioids (Broseta et al., 2002), we wished to determine whether these withdrawal signs would be sufficient to elicit alterations in pro-active/operant behavioral choices. We utilized a conditioned place aversion assay (Skoubis et al., 2001; Weitemier and Murphy, 2009) to test whether a specific environment, paired with naloxone administration during conditioning, would be avoided in favor of a different environment paired with a neutral stimulus (saline) during conditioning in chronically UV-irradiated animals. Due to the kinetics of the UV response we chose to use naloxone as it allowed an acute effect of limited duration. Naloxone induces conditioned place aversion in exogenous opioid-dependent mice (Glass et al., 2008; Kenny et al., 2006). Following conditioning, mice were permitted to move freely between the two environments and changes in place preference were measured, in the absence of additional naloxone or saline administration. Our conditioning environments were black and white boxes with dim and bright lighting, respectively, and to minimize apparatus bias we assigned the black box as the naloxone (withdrawal stimulus)-paired box and the white box as the saline (neutral stimulus)-paired box, as rodents prefer dark environments to light environments in the absence of conditioning (Roma and Riley, 2005).

We observed that chronically UV-irradiated mice conditioned with naloxone in the black box, avoided the black box in post-conditioning preference testing. Naloxone conditioning had no effect on mock-treated (non-UV irradiated) control mice, and saline conditioning in the black box had no effect on UV-irradiated or mock-treated mice (FIG. 3B). Here, naloxone was sufficient to induce conditioned place aversion in UV-irradiated mice, suggesting that chronic UV exposure imparts an opioid-like physical dependence of sufficient magnitude to guide pro-active behavior choices.

To test for the other principle feature of chronic opioid exposure, tolerance, after chronic UV treatment, we asked whether there is cross tolerance between chronic UV exposure and morphine, altering the dose required to produce analgesia (Mao et al., 2000). After chronic UV exposure, mice required significantly higher doses of morphine than mock-treated controls to achieve comparable thermal analgesia in the hot plate test, as reflected by a rightward shift in the dose-response curve and an increase in EC50 from 57 μg/kg in the mock-treated group to 270 μg/kg in the UV-exposed group (FIG. 3C). The analgesic effect of UV exposure that we detected could be a result of systemic β-endorphin acting both through the peripheral and central nervous systems, however the withdrawal effects and conditioned place aversion point to a central nervous system effect. It has been reported that radiolabeled β-endorphin peptides cross the blood-brain barrier, (Banks and Kastin 1990). To test whether it is plausible that skin-derived β-endorphin may cause central effects we decided to assess whether peripherally administered β-endorphin injected i.v. into the tail vein could cause conditioned place preference. To attempt to match an acute i.v. administered drug dose with a chronic elevation, we chose a β⁻endorphin concentration reported to cause a similar analgesic response to that which we observed in our UV exposure experiments (Tseng et al., 1976). β-endorphin or saline was injected into the tail vein of mice which were then conditioned to the white side of the CPP apparatus. The mice that had been conditioned with saline spent less time in the white box on the final day than on the initial day (FIG. 3D); this was expected as mice naturally prefer a dark environment. However, the mice that had received β-endorphin in the white box spent more time in the white box after conditioning (FIG. 3D), indicating a conditioned place preference for the environment where they experienced β-endorphin. This shows that peripherally administered β-endorphin can cause conditioned place preference, presumably through the central nervous system.

These findings show that chronic UV exposure stimulates and sustains sufficient endogenous opioid release and opioid receptor activity to develop both opioid tolerance and physical dependence.

### Example 4: Beta-Endorphin Knockout Abolishes UV Induced Behavioral Changes

To specifically examine the functional requirement for β-endorphin in these UV-associated behavioral changes, we employed β-endorphin knockout mice (lacking the C-terminus of the POMC gene) (Rubinstein et al., 1996), and found that they exhibited no significant changes in thermal or mechanical nociceptive thresholds with chronic UV exposure (FIG. 4A and FIG. 4B). The β-endorphin null mice also failed to develop signs of opioid withdrawal (FIG. 4C) and when subjected to the conditioned-place aversion test, exhibited no measurable change in place preference (FIG. 4D).

### Example 5: Keratinocyte Expression of p53 is Required for Elevated Beta-Endorphin Levels and Pain Thresholds

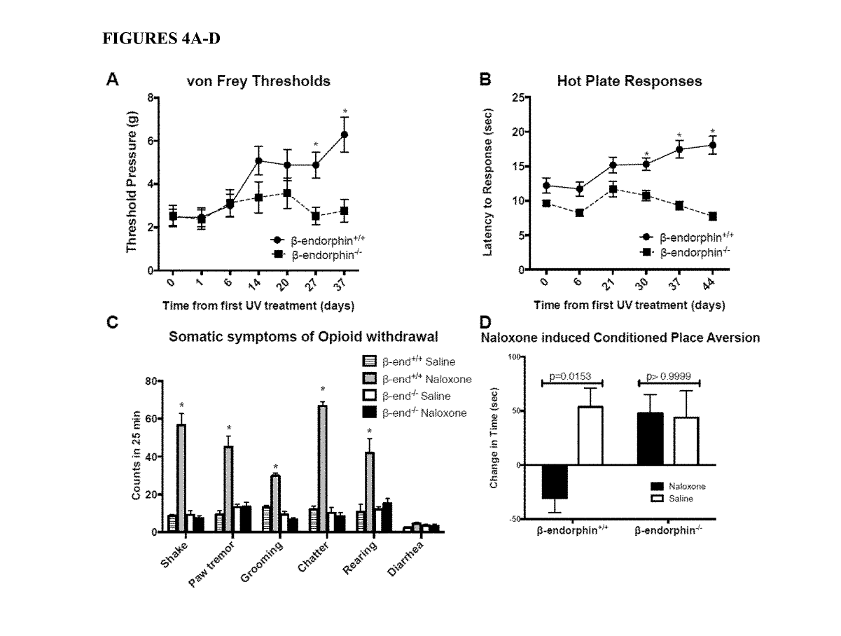

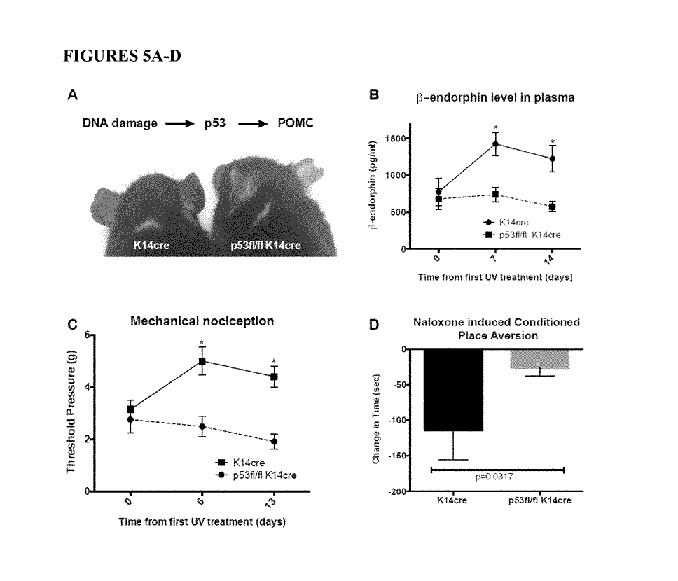

The UV induced cutaneous upregulation of POMC, the precursor to both α-MSH and β-endorphin, is mediated by the tumor suppressor p53 which directly activates POMC gene transcription in keratinocytes (Cui et al., 2007). To test whether keratinocyte expression of p53 is required for UV-mediated increases in circulating β-endorphin, we crossed a mouse strain with a foxed allele of p53 with a strain containing cre under the control of the keratin 14 promoter, which is selective to keratinocytes. We subjected the p53fl/fl K14cre and control p53+/+K14cre mice to the UV irradiation regimen and assayed plasma β⁻endorphin levels, mechanical nociception and naloxone induced conditioned place aversion. Consistent with the known role of p53 in the tanning response, there was an absence of any tanning on the ears of the p53fl/fl K14cre animals (FIG. 5A). Further we observed no increase in circulating β-endorphin (FIG. 5B) or in mechanical nociception threshold (FIG. 5C). Moreover, the K14cre control mice showed significant naloxone conditioned place aversion compared to the p53fl/fl K14cre animals (Figure D). These data indicate that keratinocyte-derived β-endorphin is a key factor in mediating UV-induced addiction.

These findings suggest that repeated UV exposure produces an opioid receptor-mediated addiction due to elevations in circulating β-endorphin, leading to increased nociceptive thresholds that are reversed by naloxone or ablated in β-endorphin null mice. Measurable withdrawal symptoms are elicited by naloxone, and pro-active place-preference behaviors were strongly induced, based on prior conditioning between opioid receptor antagonism and cage color. Further a skin specific knockout of p53, a critical step in the UV response pathway, prevented both the β-endorphin elevation and the behavioral responses.

Despite the carcinogenicity of UV and hence the serious maladaptive consequences of addiction to UV exposure, these results may also imply a potential evolutionary benefit of an endogenous mechanism that reinforces UV-seeking behavior, one that may operate by creating an opioid-mediated hedonic experience followed by dependence on the behavior to avoid the anhedonic consequences of withdrawal.

### Experimental Procedures for Examples 6-8

The following materials and methods were used in Examples 6-8.

Tissue Culture and cell Lines

UACC62 and UAC257 human melanoma cells were obtained from NCI and grown in RPMI (Cellgro) medium supplemented with 10% fetal bovine serum and penicillin/streptomycin/L-glutamine. Malme-3M human melanoma and B16 mouse melanoma cell line were obtained from ATCC and grown in DEMEM (Cellgro) medium supplemented with 5% fetal bovine serum and 1% penicillin/streptomycin/L-glutamine. Melan-a cells were kindly provided by Dorothy C. Bennett and were grown in Ham's F10 medium supplemented with 10% fetal bovine serum and 1% penicillin/streptomycin/L-glutamine. The mouse keratinocyte cell line PAM212 was generously shared by Dr. Paolo Dotto (Massachusetts General Hospital and Harvard Medical School, Boston, Mass.) and grown in 10% fetal bovine serum and 1% penicillin/streptomycin/L-glutamine. Cells were grown to 70% confluence prior to use in experiments in humidified incubators supplemented with 5% CO₂.

### RT-qPCR

After forskolin (Sigma) treatment at 20 μM final concentration at the stated times, mRNA was isolated using RNeasy® plus minikits from Qiagen, and was subjected to KAPA SYBR® FAST One-Step qRT-PCR (Kapa Biosystems). For each reaction, 100 ng of RNA was subjected to the following steps: reverse transcription for 30 min at 48° C., inactivation for 10 min at 95° C., expansion for 40 cycles (15 sec at 95° C. and 30 sec at 60° C.). The results are the average of three independent experiments. For primer sequences, refer to the primer Table 1.

TABLE 1

| Mouse Primers | Sequence 5′ to 3′ |
|---|---|
| mPOMC-qPCR-Forward | TGGCCCTCCTGCTTCAGA |
| mPOMC-qPCR-Reverse | GTCCTGGCACTGGCTGCT |
| mPOMC-qPCR-probe | [-6-FAM]CATAGATGTG |
| | TGGAGCTGGTGCCTGGA- |
| | [TAMRA-Q] |
| mGAPDH-qPCR-Forward | GGCAAATTCAACGGCACA |
| | GT |
| mGAPDH-qPCR-Reverse | AGATGGTGATGGGCTTCC |
| | C |
| mGAPDH-qPCR-probe | [6-FAM]AGGCCGAGAAT |
| | GGGAAGCTTGTCATC- |
| | [TAMRA-Q] |

siRNA Transfection

Mouse melanoma cell line (Malme-3M) was seeded in 6-well dishes and transfected with 100 pmol of double-stranded siRNA per well (0.5×10⁶ cells) using a lipidoid transfecting reagent. At 48 hrs cells were harvested and RNA was extracted. ON-TARGETplus™ SMARTpool of Si-Control and Si-MITF were bought from Dharmacon.

### Mice Blood Samples and β-Endorphin Detection Assay

Blood samples from mice were collected in EDTA microvotte tubes containing 0.6 UTI aprotinin (Sigma). Samples were spun at 3500 rpm at 4° C. for 20 min and the plasma (top layer) was isolated and transferred to a new tube and stored at −80° C. until β-endorphin measurement was performed. β-Endorphin was measured using a radioimmunoassay from Phoenix Pharmaceuticals, following the manufacturer's instructions.

### Mouse Strains and Treatment

All experiments were done in C57BL/6J (Jackson laboratory) mice. C57BL/6J Mc1r^{e/e} (described at Robbins L S, et al., Pigmentation phenotypes of variant extension locus alleles result from point mutations that alter MSH receptor function. Cell. 1993 Mar. 26; 72(6) 72, 827-834.) were crossed with K14-Scf transgenic mice as previously reported (Kunisada T, et. al., Development. 1998 August; 125(15):2915-23.). Mice were 8 weeks old at the start of each experiment.

### Mice Topical Forskolin Treatment

A crude extract of Coleus forskohlii root preparation was used as a working source of forskolin (ATZ Natural, Edgewater, N.J.) for the topical treatment of this drug (Lin C B, et. al., Modulation of microphthalmia-associated transcription factor gene expression alters skin pigmentation. J Invest Dermatol 119, 1330-1340 (2002)). The C. forskohlii extract-derived topical preparation was made by mixing the dry root powder with 70% ethanol and 30% Propyleenglycol solution for 1 hour at room temperature on a stir plate with constant agitation. Next, the solution was centrifuged (10 min, room temperature, 2,000×g) and the soluble portion (supernatant) was collected and filtered (0.45μ cellulose acetate filter). The C. forskohlii extract was stored at room temperature. Assay of content by the manufacturer (as well as independent analysis) confirmed that forskolin accounted for 20% (w/w) of the root extract in powder form. Female mice were treated with 80 μL of 20% forskolin or vehicle control daily and Male mice were pre-treated with vehicle for two weeks and then treated with forskolin (80 μL, 20%-Forskolin extract) or vehicle control daily. Basal blood samples were collected at the beginning of each experiment. After treatment started, blood samples were collected once a week until one week after treatment stopped. Samples were processed and β-endorphin was measured.

### Mice Rolipram Plus Forskolin Treatment

Female mice were treated with 40 μL of 20%-Forskolin extract daily and 40 μL of rolipram (Sigma) 20 μM in DMSO or vehicle control. Basal blood samples were collected at the beginning of each experiment. After treatment started, blood samples were collected once a week. After 4 weeks of continuous exposure to forskolin and rolipram or vehicle control, mice were injected with saline or naloxone and symptoms of opiate withdrawal were measured for 25 min post injection. Naloxone (St. Luis, Mo.) was diluted in saline at 50 mg/mL and was administrated to mice ˜200 μL, depending on the mass of each mouse (2 mg/kg). Symptoms assigned were wet dog shake (WDS), paw tremor, jumping, bouts of grooming, teeth chatter (TC), rearing and diarrhea. The occurrence in each 15 sec interval of WDS, paw tremor, bouts of grooming and teeth chatter (TC) was used to quantify these parameters. The number of fecal pellets and the individual jumping events at the end of 25 min were quantified for these two factors.

### Example 6: cAMP Increased POMC in Mouse and Human Cell Lines

MC1R^{e/e}-Red-haired mice have a frame-shift mutation in MC1R resulting in an inability to respond to α-MSH and lower amounts of eumelanin (brown/black pigment) in the skin (D'Orazio J A, N. T., Cui R, Arya M, Spry M, Wakamatsu K, Igras V, Kunisada T, Granter S R, Nishimura E K, Ito S, Fisher D E. Topical drug rescue strategy and skin protection based on the role of Mclr in UV-induced tanning. Nature 443, 340-344. (2006)). Lower ratio of eumelanin to pheomelanin gives these mice the yellow/red hair phenotype, resembling the phenotype seen in red-hair individuals. The unresponsiveness of MC1R can be rescued by bypassing the receptor with a pharmacological agent that increases cAMP, such as forskolin (Id.).

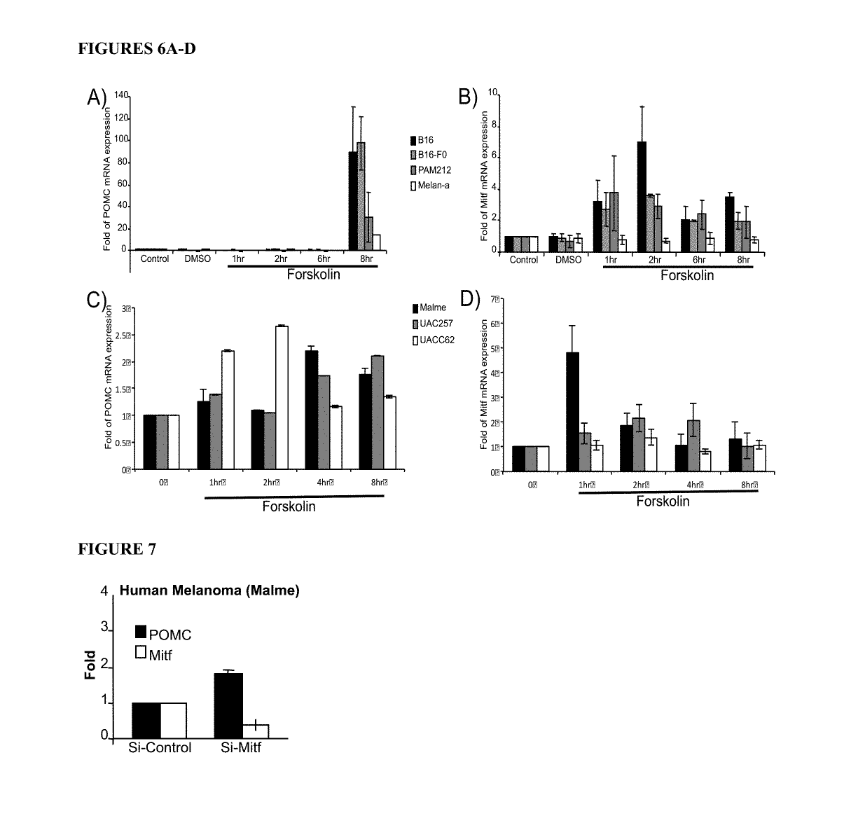

The possibility of POMC regulation by cAMP/CREB pathway was studied in-vitro by activation of cells with forskolin in a time dependent manner in 4 different mouse cell lines: B16 (mouse melanoma), B16-F0 (related mouse melanoma), Melan-a (spontaneously immortalized mouse melanocyte) (Bennett D C, C. P., Dexter T J, Devlin L M, Heasman J, Nester B. Cloned mouse melanocyte lines carrying the germline mutations albino and brown: complementation in culture. Development 105, 379-385 (1989); Bennett D C, C. P., Hart I R. A line of non-tumorigenic mouse melanocytes, syngeneic with the B16 melanoma and requiring a tumour promoter for growth. Int J Cancer 39, 414-418 (1987)) and PAM212 (Yuspa S H, H.-N. P., Koehler B, Stanley J R. A survey of transformation markers in differentiating epidermal cell lines in culture. Cancer Res 40, 4694-4703 (1980)) (mouse cancerous keratinocyte) (FIGS. 6A-6D). Treatment of mouse cells with forskolin shows a robust increase in Pomc mRNA expression in both melanocyte and keratinocyte derived cells (FIGS. 6A and 6B). We observed that Mitf mRNA expression peaks at 2 hours, while POMC mRNA expression peaks at 8 hours after forskolin treatment. This suggests an indirect mechanism for POMC induction. Alternatively it is possible that the delay in POMC upregulation (relative to MITF) could be related to post-transcriptional processes, such as RNA maturation. In human samples, we observed an increase of POMC at an earlier time point (between 2-4 hrs) after forskolin treatment (FIGS. 6C and 6D). This could represent transcriptional differences between human and mouse regulation of POMC.

In order to further study the regulation of POMC by cAMP, we transfected human melanoma cells with a luciferase construct driven by POMC promoter. Subsequently cells were exposed to forskolin for 24 hrs, but we did not observe any upregulation upon forskolin treatment (data not shown). This could be due to the nature of the construct we have used. Indeed, this construct lacks exon1, which was implicated in the CREB-dependent regulation of POMC in the pituitary-adrenal axis.

### Example 7: POMC Basal Expression is not MITF Dependent

Because in melanocytes CREB regulates MITF, we have verified the implications of this transcription factor in the cAMP-dependent regulation of POMC. In this objective, human melanoma (Malme-3M) was transfected with Si-Control or Si-Mitf. Transfection with Si-Mitf did not show a reduction on POMC expression when compared to Si-Control (FIG. 7), therefore MITF does not positively regulate basal POMC expression. However, downregulation of MITF showed an increase in POMC expression, revealing a possible repressive mechanism of POMC by MITF.

### Example 8: In-Vivo Upregulation of POMC by the cAMP Pathway Leads to an Increase in Blood Levels of Beta-Endorphin

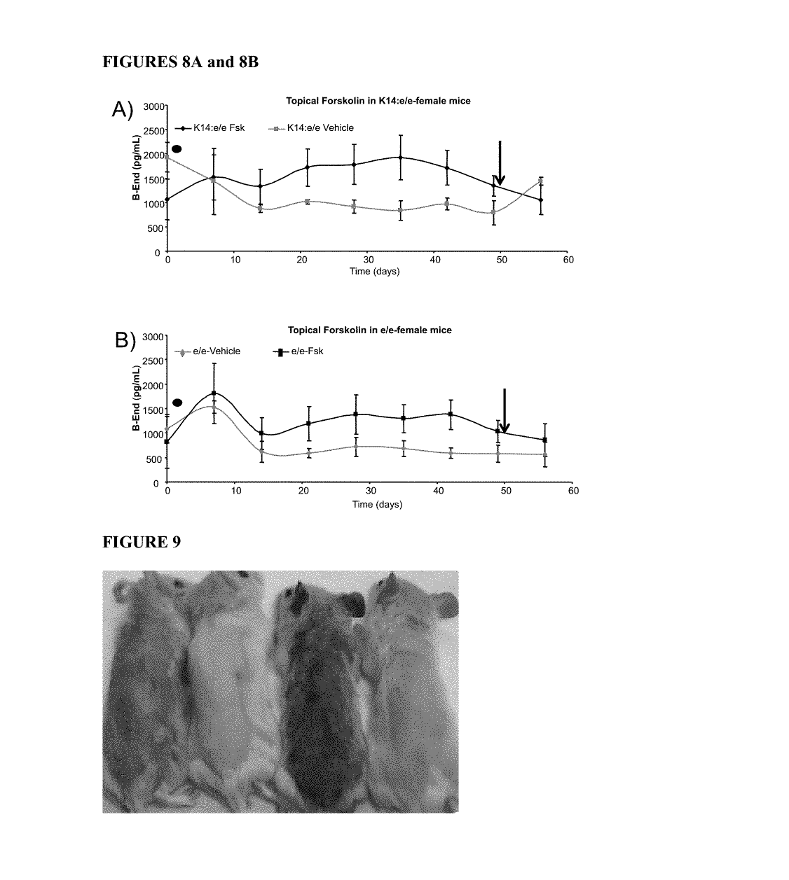

The in-vivo relevance of CREB activation of POMC was studied by looking at the response of mice to topical forskolin treatment. C57BL/6J/K14-SCF/Mc1r^{e/e} (Red-hair with epidermal melanocytes) mice and C57BL/6J/Mc1r^{e/e} (Red-hair) mice were used for all of the in-vivo treatments. In this experiment female mice were treated with topical forskolin for 8 weeks measuring the β-endorphin levels weekly. The data shows an increase in β-endorphin levels upon treatment in both K14-e/e as well as e/e female mice with no apparent difference in the induction levels (FIGS. 8A and 8B). After a month of treatment with forskolin, as expected, we saw a profound pigmentation in the K14-SCF/Mc1r^{e/e}, but not in the vehicle control or the non-K14 treated mice (FIG. 9), as they did not possess epidermal melanocytes. The color observed in the e/e-Forskolin treated mice is not pigmentation but the color of the Forskolin extract.

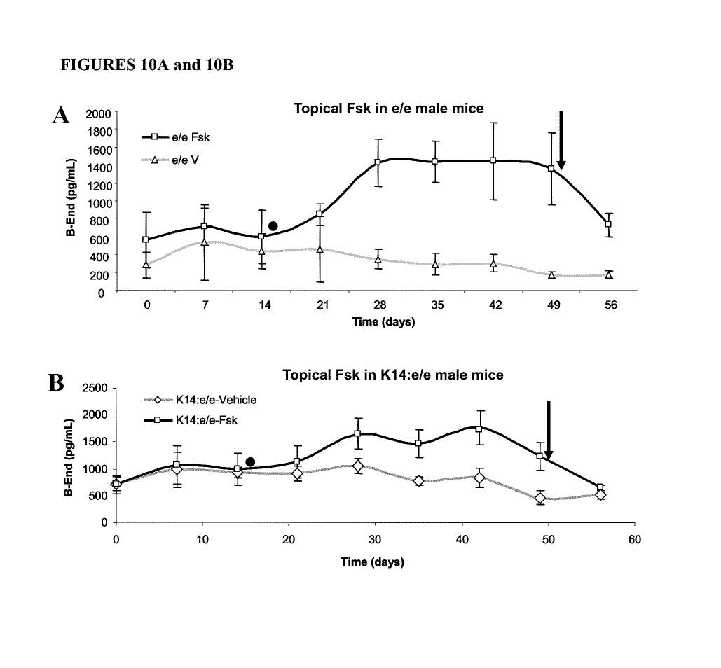

Control of β-endorphin might be different in male mice compared to females due to hormone fluctuations, so this experiment was repeated in males. Male mice also showed an upregulation of β-endorphin upon forskolin topical treatment (FIGS. 10A and 10B). These mice were subjected to a habituation treatment by daily application of vehicle control for a week, after which treatment with topical forskolin started.

We observed a higher fold induction in the non-K14-SCF mice compared to the K14-SCF/Mc1r^{e/e}, suggesting a role for the non-melanocytic lineage in the skin for this process. Also it is possible that forskolin treatment may reach the hair follicle and activate POMC in non-epidermal melanocytes. Even though the β-endorphin induction of these mice was higher, the total expression level of both groups of forskolin treated mice was equivalent. (FIGS. 10A and 10B)

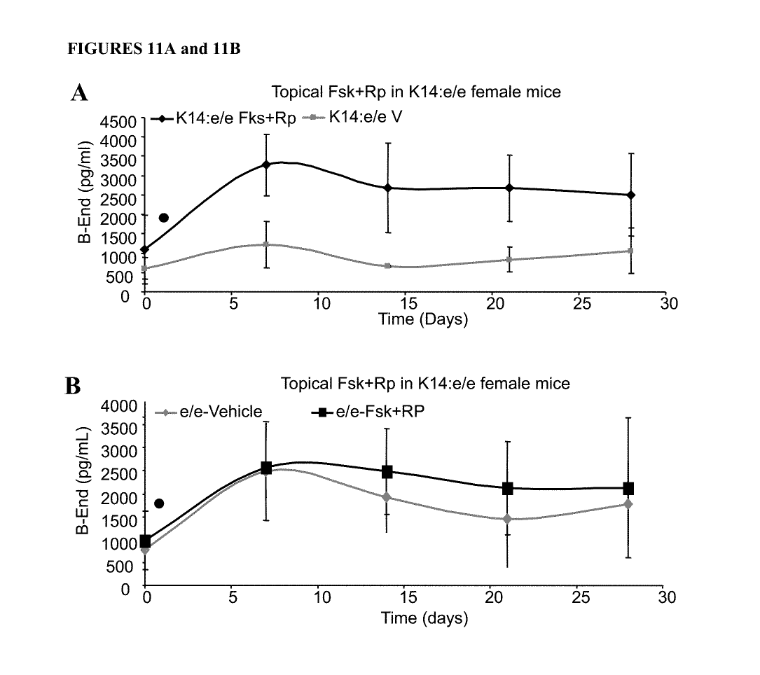

To further assess the possible involvement of the cAMP pathway in the increase of β-endorphin, we used rolipram, a drug that stimulates cAMP levels by inhibiting phosphodiesterase, an enzyme that degrades cAMP (Khaled M, L. C., Fisher D E. Control of melanocyte differentiation by a MITF-PDE4D3 homeostatic circuit. Genes Dev 24, 2276-2281 (2010); Bennett D C, C. P., Hart I R. A line of non-tumorigenic mouse melanocytes, syngeneic with the B16 melanoma and requiring a tumour promoter for growth. Int J Cancer 39, 414-418 (1987)). To induce a strong cAMP increase in mouse skin, mice were treated daily for four weeks with a combination of forskolin plus rolipram and β-endorphin was measured weekly (FIG. 11). The combinatorial treatment showed a higher increase in β-endorphin levels, compared to mice treated with forskolin alone (FIGS. 8-11). Different from the use of forskolin alone, a higher β-endorphin fold induction was observed in the K14-SCF/Mc1r^{e/e} mice compared to Non-K14, which showed no significant increase (FIG. 11).

Finally, the functional significance of systemic β-endorphin elevations upon cAMP activation was tested by carrying out behavioral studies on forskolin plus rolipram treated mice. We measured the somatic symptoms of opiate withdrawal after treatment with naloxone, an opioid antagonist (Kruger, L. (ed Lawrence Kruger) (CRC Press, Boca Raton 2001). We observed that the mice do not show any withdrawal symptoms and therefore do not show opioid dependency (Table 2). This suggests that the increase of β-endorphin observed may not have a central effect in mice.

TABLE 2

| | | | Wet | | | | | | |
|---|---|---|---|---|---|---|---|---|---|
| | | | Dog | Paw | | | Teeth | | |
| Muse | Gentype | Drug | Shake | Tremor | Jumping | oming | Chatter | Rearing | Diarrhea |
| 7 | K14:e/e | Naloxone | 145 | 0 | 0 | 17 | 80 | 0 | 0 |
| 9 | K14:e/e | Naloxone | 135 | 2 | 0 | 2 | 107 | 0 | 1 |
| 8 | K14:e/e | Saline | 150 | 1 | 0 | 9 | 51 | 1 | 0 |
| 10 | e/e | Saline | 127 | 0 | 0 | 40 | 51 | 8 | 4 |
| 12 | e/e | Naloxone | 120 | 4 | 0 | 13 | 116 | 0 | 1 |
| 13 | e/e | Naloxone | 76 | 34 | 0 | 19 | 75 | 18 | 2 |

These experiments show an upregulation of POMC 8 hrs after induction of the cAMP/CREB pathway. Even though POMC is upregulated after forskolin treatment, the results could also be explained by indirect CREB activation (a target gene of CREB activating POMC) or by a requirement of an additional transcription factor, that is needed in combination with CREB for activation of this gene.

The delayed upregulation of POMC after forskolin treatment in mouse cells (relative to MITF) suggests that a distinct mechanism (rather than simple CREB phosphorylation) is responsible, and this distinct mechanism may also help to explain why only certain tissues express POMC, since nearly all tissues experience cAMP surges from G protein coupled receptors.

Even though the fold as well as total β-endorphin induction was different, both mice expressing epidermal SCF (with epidermal melanocytes) as well as Non-SCF-K14 mice (lacking epidermal melanocytes) showed an increase in β-endorphin levels upon forskolin treatment. It is possible that the CREB mediated forskolin activation is a phenomena that happens in both: melanocytes and keratinocytes. This is supported by an in-vitro increase of POMC after forskolin treatment in both melanocytes and keratinocytes (FIGS. 6A and 6B).

The in-vivo experiments provided above showed increased blood levels of circulating β-endorphin in male as well as female mice after topical treatment with forskolin. This increase was sustained during the treatment and stopped shortly after the last forskolin application. These data were in agreement with our hypothesis that the cAMP-CREB pathway can lead to an increase of POMC expression in skin cells and therefore the release of β-endorphin in the blood stream.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating opiate withdrawal in a subject, the method comprising topically administering to a subject in need of said treatment a composition comprising an effective amount of one or more cyclic-AMP (cAMP) elevating agents.

2. A method of treating pain in a subject, the method comprising administering to a subject in need of such treatment a topical composition comprising a therapeutically effective amount of one or more cyclic-AMP (cAMP) elevating agents.

3. A method for the treatment of mood disorder in a subject, the method comprising administering to a subject in need of such treatment a topical composition comprising a therapeutically effective amount of one or more cyclic-AMP (cAMP) elevating agents.

4. The method of claim 3, wherein the cAMP elevating agent is selected from the group consisting of forskolin or a derivative thereof, amrinone, aminophylline hydrate, N6-2′-O-dibutyryl cAMP (Bu2cAMP), butein, caffeine, calmidazolium chloride, CART (61-102), cholera toxin, cicaprost, cilostamide, cilostazol, dbcAMP, (Des-Arg9,Leu8)-bradykinin, (Des-Arg9)-bradykinin, 2,6-dihydroxy-1,3-dimethylpurine, 1,3-dimethylxanthine, dobutamine, dopamine, dopexamine, DTLET, eledoisin, epinephrine, enoximone, etazolate hydrochloride, formoterol, glucocorticoid (dexamethasone), ibopamine, 4-(3-butoxy-4-methoxybenzyl)imidazolidin-2-one, imidazolium chloride, 1-[bis(4-chlorophenyl)methyl]-3-[2-(2,4-dichlorophenyl)-2-(2,4-dichlorobenzyloxy)ethyl]-1H-imidazolium chloride, 1-methyl-3-isobutylxanthine, isoproterenol, 3-isobutyl-1-methylxanthine, 8-methoxymethyl-3-isobutyl-1-methylxanthine, milrinone, α-neoendorphin, norepinephrine, neuropeptide Y fragment 22-36, papaverine hydrochloride, [Nle8,18, Tyr34]-parathyroid hormone (1-34) amide, pentoxyfilline, pertussis toxin (an AB5 protein), propentofylline, 3-methyl-1-(5-oxohexyl)-7-propylxanthine, prostaglandin E1 (PGE1), prostaglandin E2 (PGE2), prostaglandin E3 (PGE3), 3-isobutyl-1-methyl-2,6(1H,3H)-purinedione, quercetin dihydrate, rolipram, salbutamol, salmeterol, SKF 94836, [Cys3,6, Tyr8, Pro9]-substance P, theophylline, trifluoperazine dihydrochloride, TJBMX, and urotensin U.

5. The method of claim 3, wherein the one or more cAMP elevating agents is a phosphodiesterase (PDE) 4 inhibitor.

6. The method of claim 5, wherein the PDE4 inhibitor is a cAMP selective PDE4 inhibitor.

7. The method of claim 5, wherein the PDE4 inhibitor is selected form the group consisting of luteolin, cilomilast, mesembrine, rolipram, ibudilast, piclamilast, drotaverine, roflumisast, aminophylline, theophylline, 3-isobutyl-1-methylxanthine (IBMX) and caffeine.

8. The method of claim 3, wherein the one or more cAMP elevating agents comprise forskolin and rolipram.

9. The method of claim 3, further comprising irradiating the subject's skin with ultraviolet light.

10. The method of claim 9, wherein the ultraviolet light has a wavelength of between 280 and 320 nm.

11. The method of claim 10, wherein the ultraviolet light has a wavelength of between 300 and 315 nm.

12. The method of claim 3, wherein the subject has a Fitzpatrick Skin Type I, II or III.

13. The method of claim 2, wherein the pain is chronic pain or acute pain.
